(19) United States
(12) Patent Application Publication
(10) Pub. No.: US 2008/0008758 A1
(43) Pub. Date: Jan. 10, 2008

(54) Granulate-matrix

(75) Inventors: Daniel Fehr, Zurich (CH); Astrid Neidhardt, Zurich (CH); Aaldert Molenberg, Binningen (CH); Ronald Jung, Zurich (CH); Christoph Hammerle, Zurich (CH)

Correspondence Address: RISSMAN JOBSE HENDRICKS & OLIVERIO, LLP, ONE STATE STREET, SUITE 800, BOSTON, MA, 02109, US

(73) Assignee: Straumann Holding AG, Basel (CH)

(21) Appl. No.: 11/675,949

(22) Filed: Feb. 16, 2007

(30) Foreign Application Priority Data
Feb. 20, 2006 (EP) 06 003 422.0

(51) Int. Cl. A61K 9/14; A61K 31/08; A61K 31/195; A61K 31/21; A61K 31/225; A61K 31/70; A61K 33/42; A61K 38/00

(52) U.S. Cl. 424/486; 424/484; 424/489; 424/602; 514/12; 514/15; 514/23; 514/506; 514/547; 514/562; 514/723

(57) ABSTRACT

Composition comprising a granulate selected from the group consisting of autogenous bone material, bone/bone like material from natural sources, synthetic materials and mixtures thereof and a matrix obtainable by a self selective reaction of at least two precursors A and B in the presence of water. A kit for preparing said composition is also described.

### FIELD OF THE INVENTION

The present invention relates to a composition comprising a granulate and a matrix obtainable by a self selective reaction of at least two precursors forming a three dimensional network. A kit and a method for preparing said composition are also provided.

### BACKGROUND

Medical devices such as implants in general and dental implants in particular are widely used nowadays. They have become an appreciated possibility where hard tissue structures need to be fixed or replaced, e.g. in the case of bone fractures or tooth loss. However, the success of such implants strongly depends on adequate support at the implant site. If the bone mass at said site is insufficient or poor in quality, bone repair and/or bone augmentation becomes a necessity. There are different treatments applied to regain sufficient bone mass, including the use of bone graft materials of different origin, shape and size.

While there are ways to systemically treat the mass and/or strength of the bone, e.g. in osteoporosis, it is still difficult to achieve bone formation in a reliable and controllable manner. However, local bone formation would greatly benefit the adequate treatment of incidents where enhancing the bone volume is only required locally, e.g. when placing dental implants.

Methods currently used to repair bone defects include graft materials from different sources. The material is either synthetic or of natural origin. One natural graft material which is employed is autogenous bone. In contrast to bone/bone like material from natural sources (human, animals, plants, algae etc.), autogenous bone material does not trigger a strong immune response and is thus not rejected by the host. However, autogenous bone material requires a second surgery for harvesting the bone increasing the risk of unwanted infection and/or inflammation at this site and significantly increases treatment costs. Further, the removal of bone material leads, at least temporarily, to a weakened structure at this site and causes a painful healing process.

During the last years it became more and more clear that the use of various bioactive factors improves bone repair and/or bone augmentation. It has also been shown that the method of application of such factors greatly influences their regenerative effect. Despite continuous efforts to develop methods for the controllable presentation and release of said factors, this is still one of the common problems in this field.

In the state of the art, different biomaterials for tissue augmentation or release of bioactive factors have been described.

WO 00/44808 discloses a polymeric biomaterial formed by nucleophilic addition reactions to conjugated unsaturated groups. The obtained biomaterial, which is in the form of a hydrogel, may be used for example as glues or sealants and as scaffolds for is tissue engineering and wound healing applications. Also said hydrogels degrade fast under physiological conditions.

U.S. Pat. No. 5,626,861 discloses a method for the fabrication of a macroporous matrix that may be used as implant material. The composites are formed from a mixture of biodegradable and biocompatible polymer which is dissolved in an organic solvent such as methylene chloride or chloroform and then mixed with hydroxyapatite. The latter is a particulate calcium phosphate ceramic. The material has irregular pores in the size range between 100 and 250 microns. Bioactive factors may be non-covalently incorporated in the composite.

U.S. Pat. No. 5,204,382 describes injectable implant compositions comprising a biocompatible ceramic matrix mixed with an organic polymer or collagen suspended in a fluid carrier. The ceramic particles are in the size range of 50 μm to 250 μm.

U.S. Pat. No. 6,417,247 discloses polymer and a ceramic matrix. The compositions are normally liquid and harden upon a certain stimulus, e.g. elevated temperatures.

WO 2004/103421 describes a hydroxylapatite/silicon dioxide material having a defined morphology. A highly porous bone substitute material based on the hydroxylapatite/silicon dioxide material is also described.

WO 03/040235 discloses a synthetic matrix for controlled cell ingrowth and tissue regeneration. The matrix comprises a three-dimensional polymeric network formed by multi-functional precursors.

WO 2004/054633 describes a macroporous synthetic ceramic which can be used to produce granulated bone substitute material.

EP 0 324 425 discloses a method for producing a medical bone prosthesis using at least one of α-tricalcium phosphate and tetracalcium phosphate.

US 2004/0019132 describes methods and compositions for manufacturing a bone graft substitute. A powder compaction process is used to generate a shaped product comprising granulated bone material, such as demineralized bone matrix.

WO 03/092760 discloses a structured composite as a carrier for the tissue engineering and implant material of bones, consisting of a mass of porous calcium phosphate granulates.

WO 2006/072622 describes supplemented matrices comprising a PTH releasably incorporated therein, optionally containing a granular material.

As used herein, the words “polymerization” and “cross-linking” are used to indicate the linking of different precursors to each other to result in a substantial increase in molecular weight. “Cross-linking” further indicates branching, typically to obtain a three dimensional polymer network.

By “self selective” is meant that a first precursor A of the reaction reacts much faster with a second precursor B than with other compounds present in the mixture at the site of the reaction, and the second precursor B reacts much faster with the first precursor A than with other compounds present in the mixture at the site of the reaction. The mixture may contain other biological materials, for example, drugs, peptides, proteins, DNA, RNA, cells, cell aggregates and tissues.

By “conjugated unsaturated bond” the alternation of carbon-carbon, carbon-heteroatom or heteroatom-heteroatom multiple bonds with single bonds is meant. Such bonds can undergo addition reactions.

By “conjugated unsaturated group” a molecule or a region of a molecule, containing an alternation of carbon-carbon, carbon-heteroatom or heteroatom-heteroatom multiple bonds with single bonds, which has a multiple bond which can undergo addition reactions is meant. Examples of conjugated unsaturated groups include, but are not limited to acrylates, acrylamides, quinines, and vinylpyridiniums, for example 2- or 4-vinylpyridinium.

### SUMMARY OF THE INVENTION

The problem of the present invention is to provide a bone repair and/or bone augmentation material which has an excellent biocompatibility and mechanical stability allowing in situ repair of the bone defect and/or bone augmentation while minimizing the risk of unwanted inflammation, eliminating the need for second surgery for harvesting autogenous bone material and not bearing the risk of infection. In addition, the treatment costs are significantly reduced.

The composition according to one embodiment of the present invention comprises a granulate and a degradable polymeric matrix. Several cross-linked substances are known in the art, which are able to provide a porous three-dimensional biodegradable matrix suitable for tissue regeneration and obtainable by a self selective reaction. An example for a polymeric material is PEG.

In one preferred embodiment, such polymeric matrix is obtained by a self selective reaction of two or more precursors, as defined below, in the presence of water. The combination of said granulate and said matrix yields a composition having excellent bone repair and/or bone augmentation properties. The combination of said matrix with said granulate synergistically improves the bone repair and/or bone augmentation. While the matrix provides a three-dimensional scaffold, the granulate ensures a good mechanical stability. Since precursors forming the matrix and granulate are preferably mixed just prior to use, an optimal distribution of the granulate throughout the entire composition can be achieved. The precursors, which are the monomers forming the matrix, are soluble in water. It is important to note that precursors and not polymers are mixed with the granulate allowing the formation of the matrix in situ. Consequently, the aqueous solution comprising the precursors and the granulate is not viscous and can be rapidly mixed without difficulties. The rapid generation of the matrix preserves the optimal distribution of the granulate and avoids imbalances due to possible sedimentation of the granulate.

Furthermore, the combination of a hydrogel matrix and granulate allows modelling of the granular putty to the desired shape, stabilizes the shape and prevents granulate migration.

If appropriate, a viscosity modifier, such as CMC (carboxymethylcellulose), PGA (propylene glycol alginate) or Xanthan, can be added to ensure optimal physical properties for administration in situ, e.g. in case a relatively large amount of liquid should be added to the granules. Thus, uniform and optimal bone repair and/or bone augmentation properties are ensured throughout the entire three-dimensional structure formed by the composition.

In previously known treatments, the bone filler material is applied upon mixing with non polymerizing liquids, e.g. NaCl solutions or blood. As a result, the administered bone grafting mixture may not provide for an accurate stability required for successful new hard tissue formation. The bone graft material is usually exposed to mechanical stress due to the overlying layer of soft tissue or other impacts, which can lead to the deformation, migration or even collapse of the augmentate.

The composition of the present invention will overcome this problem by the combination of an appropriate filler material, e.g. calcium phosphate granulate, and a polymeric matrix, e.g. PEG, and thereby provide for controlled and safe bone repair and/or bone augmentation.

Apart from the simple handling, the single components of the composition, the precursors forming the matrix and the granulate, have an excellent stability and thus a long shelf life. Advantageously, the components are stored in a dry form, e.g. as a powder, and the precursors are dissolved immediately prior to application. Alternatively, the components may be stored in solvents that protect their functionalities.

Further, in various embodiments the composition is biodegradable thereby leaving space for natural bone to grow into. Again, this avoids surgery in order to remove remaining parts of the bone repair and/or bone augmentation material subsequently to the completed healing of the bone defect. The degradation products are easily excreted and non-toxic.

The granulate serves on one hand as a filler expanding the volume of the composition and, on the other hand, it provides the necessary mechanical strength of the composition. Furthermore, it preferably offers a scaffold surface for bone deposition. There is a wide variety of materials which can be employed as granulate, e.g. bone materials or synthetic materials. Examples of granulate materials are autograft bone, hydroxyapatite, tricalcium phosphate and mixtures thereof.

Further examples of granulate materials include autogenous bone materials such as chin, retromolar and nasal spine (all harvested intraorally), crista, iliaca and calotte (all harvested extraorally), bone/bone like materials from natural sources such as freeze dried bone allograft (FDBA), demineralized freeze dried bone allograft (DFDBA; Grafton®), bovine material (BioOss®, Osteograph®, Navigraft®, Osteograft®), coralline material (Pro Osteon®, Interpore 500®), algae material (Frios Algipore®), and collagens. Synthetic materials are hydroxyapatite (Ostim®), tricalciumphosphate (Cerasorb®, BioResorb®, Ceros® etc.), mixtures of hydroxyapatite and tricalciumphosphate (Straumann BoneCeramic®), bioactive glass (PerioGlas®, Biogran®), calcium sulfate and carbonated apatite.

The synthetic materials provide the advantage that they are of non-animal origin, thus eliminating the possible risk of infection with human or animal pathogens, depending on the source of the natural materials, which is always present when not autogenous bone material but bone/bone like materials from natural sources are used. In addition, synthetic granulates eliminate the need for a second surgery, in contrast to the case when autogenous bone material is employed. Said second surgery can be a prominent source of complications and additional costs. Apart from the fact that sound bone structures are at least temporarily weakened, infections or inflammation may occur, further complicating the healing process of the surgery site which itself is already painful.

Another advantage of synthetic materials is that its manufacturing allows for control of parameters such as chemical composition, crystallinity, and porosity.

Below, precursors A and B forming the matrix are described in more detail.

The first precursor A comprises a core which carries n chains with a conjugated unsaturated group or a conjugated unsaturated bond attached to any of the last 20 atoms of the chain. In a preferred embodiment said conjugated unsaturated group or conjugated unsaturated bond is terminal. The core of the first precursor A can be a single atom such as a carbon or a nitrogen atom or a small molecule such as an ethylene oxide unit, an amino acid or a peptide, a sugar, a multifunctional alcohol, such as pentaerythritol, D-sorbitol, glycerol or oligoglycerol, such as hexaglycerol. The chains are linear polymers or linear or branched alkyl chains optionally comprising heteroatoms, amide groups or ester groups. Beside the chains, the core of precursor A may be additionally substituted with linear or branched alkyl residues or polymers which have no conjugated unsaturated groups or bonds. In a preferred embodiment the first precursor A has 2 to 10 chains, preferably 2-8, more preferably 3-8, most preferably 4-8 chains. The conjugated unsaturated bonds are preferably acrylates, acrylamides, quinines, 2- or 4-vinylpyridiniums, vinylsulfone, maleimide or itaconate esters of formula Ia or Ib 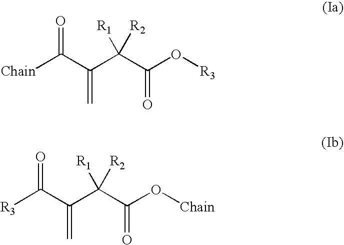
wherein R₁ and R₂ are independently hydrogen, methyl, ethyl, propyl or butyl, and R₃ is a linear or branched C₁ to C₁₀ hydrocarbon chain, preferably methyl, ethyl, propyl or butyl.

The second precursor B comprises a core carrying m chains each having a thiol or an amine group attached to any of the last 20 atoms at the end of the chain. For example a cysteine residue may be incorporated into the chain. Preferably the thiol group is terminal. The core of the second precursor B can be a single atom such as a carbon or a nitrogen atom or a small molecule such as an ethylene oxide unit, an amino acid or a peptide, a sugar, a multifunctional alcohol, such as pentaerythritol, D-sorbitol, glycerol or oligoglycerol, such as hexaglycerol. The chains are linear polymers or linear or branched alkyl chains optionally comprising heteroatoms, esters groups or amide groups. In a preferred embodiment the second precursor B has 2 to 10 chains, preferably 2-8, more preferably 2-6, most preferably 2 to 4 chains.

In a preferred embodiment, the core of precursor B comprises a peptide which comprises one or more enzymatic degradation sites. Examples for enzymatic degradation sites are substrate sequences for plasmin, matrix metallo-proteinases and the like.

In a preferred embodiment, precursor A and/or B comprises a peptide which comprises one or more enzymatic degradation sites. Precursor A and/or B can also be a peptide comprising 2 cysteine residues and one or more enzymatic degradation sites. Such precursors are described in WO 03/040235 which is incorporated herein by reference. Examples for enzymatic degradation sites are substrate sequences for plasmin, matrix metallo-proteinases and the like.

In a preferred embodiment a precursor which comprises a peptide or is a peptide comprising 2 cysteine residues and one or more enzymatic degradation sites as described for precursor B can be used as a third precursor.

The first precursor A compound has n chains, whereby n is greater than or equal to 2, and the second precursor B compound has n chains, whereby m is greater than or equal to 2. The first precursor A and/or the second precursor B may comprise further chains which are not functionalized. The sum of the functionalized chains of the first and the second precursor, that means m+n, is greater than or equal to 5. Preferably the sum of m+n is equal to or greater than 6 to obtain a well formed three-dimensional network.

The precursors forming the matrix are preferably dissolved or suspended in aqueous solutions. The precursors do not necessarily have to be entirely water-soluble.

The granulate can be wetted with the precursor solutions or suspended in a larger amount of precursor solutions.

Since no organic solvents are necessary, preferably only aqueous solutions and/or suspensions are present. These are easy to handle and do not require any laborious precautions as might be the case if organic solvents are present. Further, organic solvents may be an additional risk for the health of the staff and the patients exposed to these solvents. The present invention eliminates this risk.

The use of at least two precursors which form a three dimensional network by a self selective reaction can advantageously be applied in situ. This means, the composition can be brought to the site of the bone defect in the form of a liquid or paste, allowing a precise control of the amount of composition applied. The still liquid composition optimally adopts the shape of the bone defect, ensuring optimal fit and hold. Furthermore, it allows modeling of the composition to the desired shape. No further fixation is needed. The hardening of the composition can be completed within minutes, starting at the time of mixing. It preferably does not require any complicated triggering stimulus and the self selectivity of the reaction is such that surrounding tissue is not harmed.

In a preferred embodiment the granulate comprises calciumphosphate, which is highly biocompatible in terms that it is inert, i.e., does not elicit inflammatory processes or further unwanted biological reactions.

In a further preferred embodiment the granulate comprises hydroxyapatite (HA) and/or tricalciumphosphate (TCP).

In a preferred embodiment the composition comprises a granulate wherein the weight ratio of hydroxyapatite/tricalciumphosphate in the granulate is between 0.1 to 5.0, preferably between 1.0 to 4.0, and most preferably between 1.0 to 2.0.

In another preferred embodiment the content of hydroxyapatite (HA) in the granulate is at least 1% by weight, preferably equal to or more than 15% by weight, and most preferably equal to or more than 50% by weight.

The mechanical strength of the composition is greatly influenced by the amount of granulate present in the composition. Good results are achieved with compositions comprising 10% to 80% by weight granulate. Preferred is the range of 20% to 70% and most preferred is the range of 30% to 60%.

In a further preferred embodiment the conjugated unsaturated group or the conjugated unsaturated bond of first precursor A is an acrylate, a quinine, a 2- or 4-vinylpyridinium, vinylsulfone, maleimide or an itaconate ester of formula Ia or Ib.

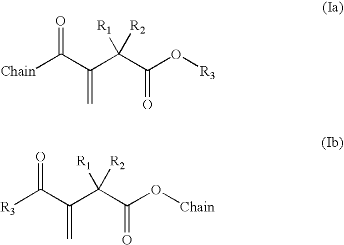

Most preferred are acrylates.

In a particularly preferred embodiment precursor A is chosen from the group consisting of

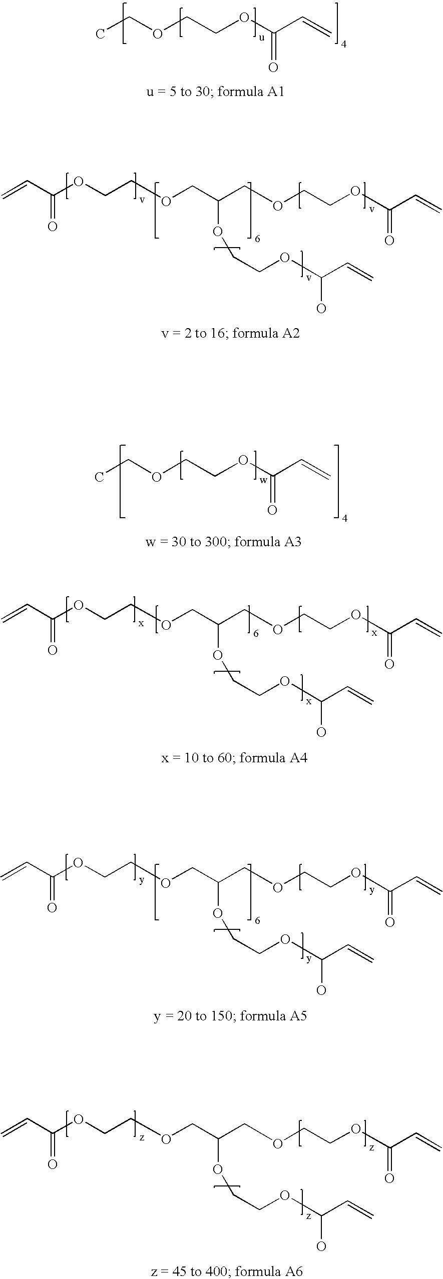

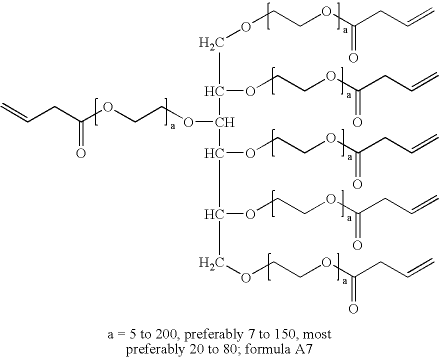

In another preferred embodiment precursor B comprises a thiol moiety or is selected from the group consisting of

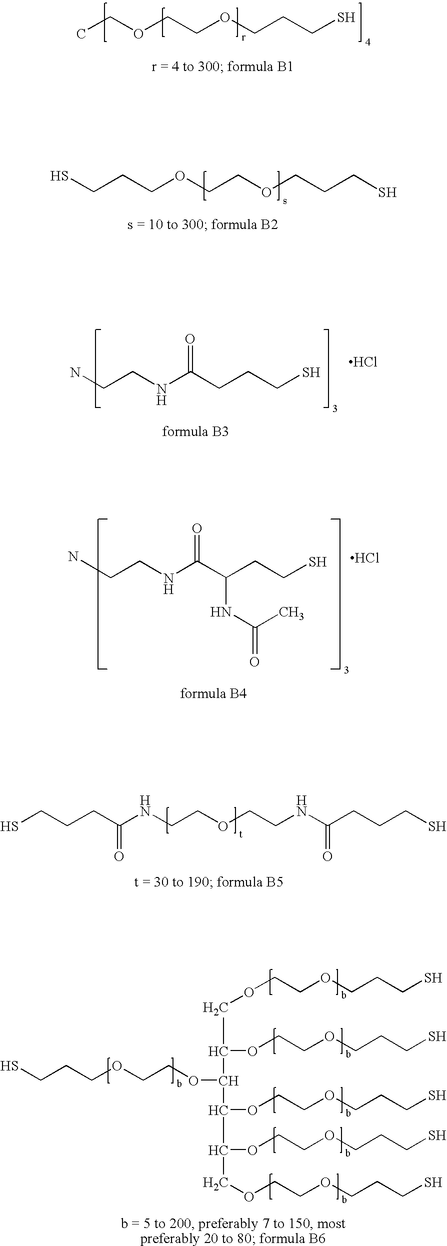

Most preferred precursor A is a PEG-acrylate carrying 4 chains and having a molecular weight of approximately 15,000 Da. Most preferred precursors B are selected from the group consisting of a linear PEG-dithiol having a molecular weight of approximately 3500 Da and PEG-thiol carrying 4 chains and having a molecular weight of about 2400 Da.

Precursor A and/or B can significantly vary in their molecular weight, preferably in the range of 500 Da to 100,000 Da, more preferably in the range of 1000 to 50,000 and most preferably in the range of 2000 to 30,000.

In a preferred embodiment the chains of precursor A and/or B are a polymer selected from the group consisting of poly(vinyl alcohol), poly(alkylene oxides), poly(ethylene glycol), poly(oxyethylated polyols), poly(oxyethylated sorbitol, poly(oxyethylated glucose), poly(oxazoline), poly(acryloyl-morpholine), poly(vinylpyrrolidone), and mixtures thereof. In a particularly preferred embodiment the chains of precursor A and/or B are poly(ethylene glycol). The poly(ethylene glycol) can be either linear or branched.

In another preferred embodiment precursor A is used with a precursor B which is a peptide comprising 2 cysteine residues and one or more enzymatic degradation sites. The cysteine residues are preferably located at the terminus of the peptide.

In a preferred embodiment the composition comprises at least one bioactive factor. The bioactive factor can be added when mixing the other components of the composition. If the bioactive factor does not comprise a reactive group, e.g. a thiol or an amine group, said bioactive factor will not be covalently bound to the matrix, but simply be entrapped in the composition. The bioactive factor is then released by diffusion. However, the factor may also be covalently bound to the matrix, e.g., this can be achieved by a thiol moiety present in the bioactive factor which reacts with the conjugated unsaturated group or bond present in precursor A upon mixing. A thiol moiety is preferably present, e.g. in the amino acid cysteine. This amino acid can easily be introduced in peptides, oligo-peptides or proteins. It is also possible to adsorb the bioactive factor on the granules prior to the mixing of the granules with solutions comprising the first precursor A and the second precursor B.

In a preferred embodiment the bioactive factor is selected from the group consisting of parathyroid hormones (PTH), peptides based on PTH, peptide fragments of PTH, peptides comprising an RGD tripeptide, transforming growth factor beta family (TGFβ), bone morphogenetic protein family (BMP), platelet derived growth factor family (PDGF), vascular endothelial growth factor family (VEGF), insulin like growth factor family (IGF), fibroblast growth factor family (FGF), enamel matrix derivative proteins and peptides (EMD) as described in EP 01165102 B1, prostaglandin E₂ (PGE₂) and EP2 agonists, and dentonin. Dentonin is a peptide fragment of matrix extracellular phosphoglycoprotein (MEPE) found in bone and dental tissues. It is further described in WO 02/14360. Also, extracellular matrix proteins, such as fibronectin, collagen, and laminin, may be used as bioactive factors. These peptides and proteins may or may not comprise additional cysteine. Such cysteine facilitates the covalent attachment of the peptides and proteins to the matrix.

In another preferred embodiment the bioactive factor is selected from the group consisting of parathyroid hormones (PTH), peptides based on PTH and peptide fragments of PTH. Parathyroid hormones have been shown to exert multiple anabolic effects on bone tissue. Particularly preferred is a peptide comprising the first 34 amino acids of PTH. This peptide may or may not contain an additional cysteine, which facilitates the covalent attachment of the peptide to the matrix. Such peptides can be produced by enzymatic cleavage of PTH or by peptide synthesis. In a further preferred embodiment the bioactive factor is selected from the group consisting of amelogenin, amelin, tuftelin, ameloblastin, enamelin and dentin sialoprotein.

The effectiveness of the matrix can be enhanced by introduction of cell attachment sites. For example, the RGD sequence motif plays an important role in specific cell adhesion. A possible cell attachment peptide is H-Gly-Cys-Gly-Arg-Gly-Asp-Ser-Pro-Gly-NH₂, which can be covalently attached to the matrix through its cysteine.

The bioactive factors may be prepared from natural sources, by synthetic or recombinant means or a mixture thereof.

The present invention also relates to kits used to prepare a composition according to the present invention. The kit comprises (i) a granulate, (ii) a precursor A and (iii) a precursor B which are each individually stored. The kit may also comprise more than one granulate and more than two precursors.

In a preferred embodiment the kit also comprises at least one bioactive factor as a further component (iv) which is individually stored as well. If desired, the kit may comprise two or more bioactive factors stored as a premix or, preferably, individually stored. In the latter case, the factors can be mixed when the kit is used according to specific needs of the patient.

It is also possible that the kit comprises certain components in premixed form. For instance, the granulate and precursor A can be stored as premix, the granulate and precursor B can be stored as premix and also precursor B and the bioactive factor can be stored as premix. The precursors can be stored in dry form or in a suitable solvent (e.g. 0.04% acetic acid). A suitable buffer solution can be added immediately prior to application. The precursors are preferably stored in a dry form. The bioactive factor can be (pre-)adsorbed to the granulate. Further, the bioactive factor can be stored in a dry (lyophilized) form or in an aqueous solution which is suitably buffered. The former provides excellent stability and thus a long shelf life, the latter provides a very user-friendly handling.

A method for preparing a composition according to the present invention is also provided. For this purpose, the granulate, the precursor A and the precursor B are mixed in the presence of water. Preferably, the water is buffered near or at the physiological pH. A suitable buffering range for the matrix is pH 7.4 to 9.0. The polymerization preferably starts upon mixing of the different components and a hydrogel is formed within a quite short period of time (10 seconds up to 10 minutes). The precursors do not necessarily have to be completely water soluble.

The mixing of the different components can be achieved in several ways. If the precursors A and B are stored as aqueous solutions they can be mixed with the granulate by means of a suitable mixing device. Preferably they are filter sterilized just prior to their use. Most preferably, the components are sterilized at the time of production and packed in such a way that sterility is preserved. If the components are stored in powder form, they can each be dissolved in an appropriate buffered aqueous solution.

If the kit comprises a bioactive factor, the factor may be premixed or pre-reacted with any of the precursors or added separately in dry or lyophilized form or dissolved state. For instance, if the bioactive factor comprises a thiol, it can be pre-reacted with precursor A. The bioactive factor can also be preadsorbed to the granulate prior to mixing with the precursors A and B.

The present invention also relates to the use of the composition as material for bone repair and/or bone augmentation.

In a preferred embodiment the composition according to the present invention is used as bone repair and/or bone augmentation material.

### BRIEF DESCRIPTION OF THE DRAWINGS

FIG. 1 shows a mandibular defect model with granular putty after gelation, applied by surgeon 1;

FIG. 2 shows the swelling of the hydrogel samples (9.8 wt % PEG with/without granules) against time in PBS (pH 7.4) at 37° C.; average values of 6 samples (±SD) are given;

FIG. 3 shows the area of bone regeneration for the rabbit cranial cylinder model applying granules with PEG; values are displayed as box-plots ranging from the 25^{th} to the 75^{th} quantiles, including the median and whiskers extending 1.5 times the interquartile range; and

FIG. 4 shows the percentages of mineralized bone found for the rabbit cranial cylinder model applying granules with PEG; values are displayed as box-plots ranging from the 25^{th} to the 75^{th} quantiles, including the median and whiskers extending 1.5 times the interquartile range.

### DETAILED DESCRIPTION

### Example 1

164 mg (0.084 mmol thiol) of HS-PEG-SH 3.4 k (Nektar, Huntsville, Ala., USA) were dissolved in 1.71 ml of 0.05% acetic acid and 326 mg (0.083 mmol acrylate) of 4-arm PEG-acrylate 15 k (Nektar, Huntsville, Ala., USA) were dissolved in 1.55 ml of 0.05% acetic acid containing 100 ppm of methylene blue. Mixing aliquots of both PEG solutions with a 0.4 M triethanolamine/HCl buffer (pH 8.85) in a volume ratio of 1.5:1.5:1 yielded a gel in 3.5 minutes at 25° C.

Aliquots of the three solutions (V_{PEG-thiol}:V_{PEG-acrylate}:V_{buffer}=1.5:1.5:1) were pipetted to HA/TCP (60%/40%) granules (Straumann Bone Ceramic, Institut Straumann AG, Basel, Switzerland) and mixed. Three surgeons independently evaluated the application properties of compositions with various granules/liquid ratios:

| Granules | Liquid | | | |
|---|---|---|---|---|
| (g) | (ml) | Surgeon 1 | Surgeon 2 | Surgeon 3 |
| 0.5 | 0.6 | good consistency | slightly too | good application |
| | | | little liquid | properties |
| 0.5 | 0.7 | best consistency, | good | good application |
| | | all liquid is | application | properties |
| | | absorbed | properties | |
| 0.5 | 0.9 | good consistency, | — | — |
| | | some liquid not | | |
| | | absorbed | | |

The tests showed that the granules readily absorbed the PEG solution and the resulting granular putty was easy to apply in a mandibular defect model and yielded a stable augmentate after gelation of the PEGs.

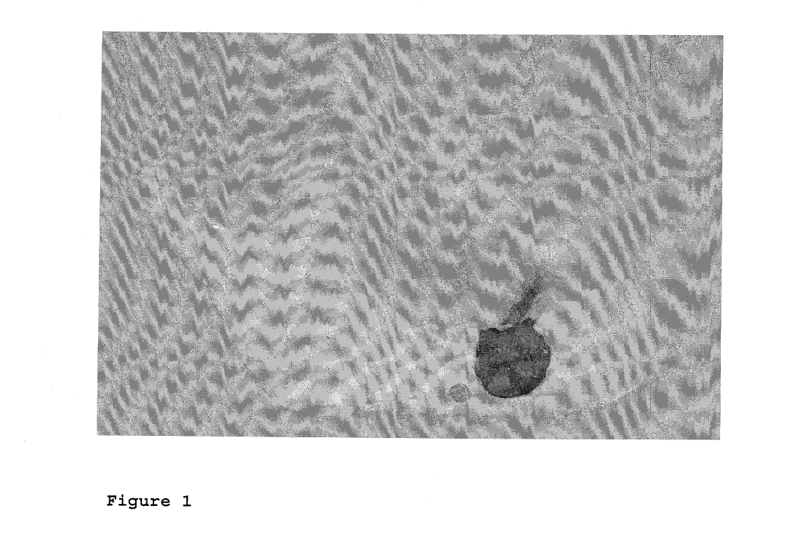

FIG. 1 shows a mandibular defect model with granular putty after gelation, applied by surgeon 1.

### Example 2

Formulation 1:

150 mg (0.47 mmol acrylate) of 8-arm PEG-acrylate 2 k were dissolved in 0.60 ml of 0.02 M triethanolamine/HCl buffer (pH 7.6) and 311 mg (0.49 mmol thiol) of 4-arm PEG-thiol 2 k were dissolved in 0.44 ml of water.

Mixing equal aliquots of both solutions yielded a gel in ca. 35 seconds at 37° C.

Formulation 2:

170 mg (0.45 mmol acrylate) of 6-arm PEG-acrylate 2 k were dissolved in 0.58 ml of 0.05 M triethanolamine/HCl buffer (pH 9.8) and 190 mg (0.47 mmol thiol) of 6-arm PEG-thiol 2 k were dissolved in 0.56 ml of water.

Mixing equal aliquots of both solutions yielded a gel in ca. 75 seconds at 37° C.

Formulation 3:

69 mg (0.018 mmol acrylate) of 4-arm PEG-acrylate 15 k were dissolved in 0.131 ml of 0.04% aqueous acetic acid containing 100 ppm methylene blue and 11 mg (0.018 mmol thiol) of 4-arm PEG-thiol 2 k were dissolved in 0.189 ml of 0.04% aqueous acetic acid.

Mixing aliquots of both PEG solutions with a 0.05 M triethanolamine/HCl buffer (pH 8.7) in a volume ratio of 1:1:3 yielded a gel in ca. 2.5 minutes at 25° C.

Mixing any of the above 3 formulations with HA/TCP (60%/40%) granules (Straumann Bone Ceramic, Institut Straumann AG, Basel, Switzerland) yields a granular putty with similar application properties as those of the formulation of example 1.

### Example 3

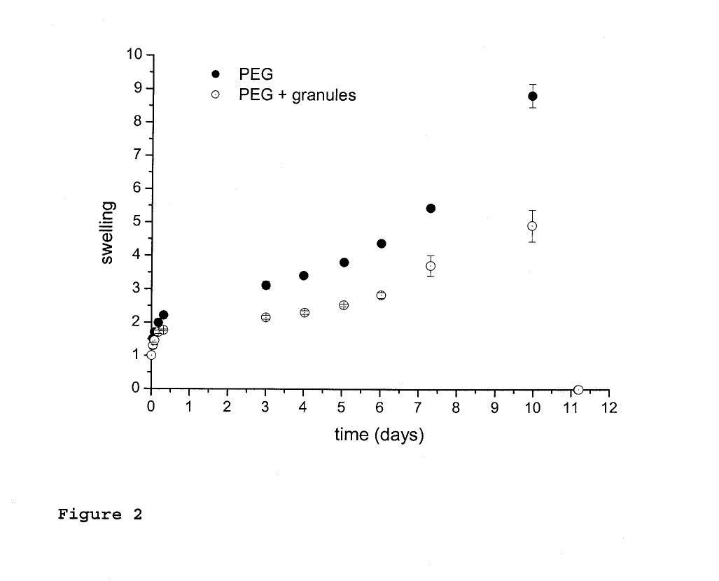

A 0.1 M aqueous solution of triethanolamine was brought to pH 8.7 using 2 M hydrochloric acid. 4-arm PEG-acrylate 15 k and HS-PEG-SH 3.4 k (both from Nektar, Huntsville, Ala., USA) were dissolved in this buffer solution, such that the total PEG concentration was 9.8 wt % and equimolar amounts of acrylate and thiol groups were present. Half of the solution was mixed with HA/TCP (60%/40%) granules in a ratio of 0.6 ml liquid per 0.5 g granules. From both the PEG solution and the mixture of PEG solution with granules, 6 cylindrical gels with a diameter of 6 mm were cast using stainless steel molds. After curing for 15 min, the gels were weighed, added to a Falcon tube containing 10 ml of 30 mM PBS (pH 7.4) and placed in a water bath at 37° C. At regular intervals the gels were taken from the buffer solution, blotted dry, and weighed. The pH of the buffer solution was checked and, if the value deviated by more than 0.1 from pH 7.4, the buffer was replaced by fresh 30 mM PBS (pH 7.4). The disintegration of the gels was followed by dividing their weight at each time point by the weight immediately after casting. Both the gels with and those without granules degraded at the same rate and had completely degraded within ca. 11 days (FIG. 2), however, the addition of granules led to a markedly lower swelling.

FIG. 2 shows the swelling of the hydrogel samples (9.8 wt % PEG with/without granules) against time in PBS (pH 7.4) at 37° C. Average values of 6 samples (±SD) are given.

### Example 4

Methods

16 adult (12 months old) New Zealand White rabbits, weighing between 3 and 4 kg, were anesthetized and obtained each 4 titanium cylinders of 7 mm in height and 7 mm in outer diameter, which were screwed in 1 mm deep circular perforated slits made in the cortical bones of the calvaria. The following 4 treatment modalities were randomly allocated: (1) empty control, (2) a combination of PEG matrix and hydroxyapatite (HA)/tricalciumphosphate (TCP) granules (Straumann Bone Ceramic; Institut Straumann AG, Basel, Switzerland), and a combination of PEG matrix containing either 100 (3) or 20 μg/g gel (4) of PTH₁₋₃₄ and HA/TCP granules. Immediately before application, 4-arm PEG-acrylate 15 k and HS-PEG-SH 3.4 k (both from Nektar, Huntsville, Ala., USA) were each dissolved in a 0.1 M aqueous triethanolamine/HCl buffer (pH 8.7), such that the total PEG concentration in both solutions together was 9.8 wt % and equimolar amounts of acrylate and thiol groups were present. Both PEG solutions were then sterile filtered. For the activated gels, a 35 amino acid peptide of the parathyroid hormone (cys-PTH-₁₋₃₄) and a 9 amino acid cys-RGD peptide (both from Bachem, Bubendorf, Switzerland) were additionally added to the PEG-acrylate solution, resulting in the formation of covalent bonds between the cystein-residues and the PEG-acrylate. The final concentrations for the peptides were 350 μg/g gel for cys-RGD and 20 or 100 μg/g gel for cys-PTH₁₋₃₄.

The PEG solutions were then applied onto the HA/TCP granules and mixed for about 10 seconds. Subsequently, this granular putty was applied into the determined cylinders. Within 60 seconds, the PEG gels set and thus stabilized the HA/TCP granules. The cylinders were left open towards the bone side but were closed with a titanium lid towards the covering skin-periosteal flap. The periosteum and the cutaneous flap were adapted and sutured for primary healing.

After 8 weeks, the animals were sacrificed and ground sections were prepared for histology.

The bone formation in the cylinders was evaluated histologically. Mean values and standard deviations were calculated for the amounts of bone formation within the cylinders, either evaluated by the point measurements or by the area of bone regeneration and for the graft to bone contact. For statistical analysis, repeated measures ANOVA and subsequent pairwise Student's t-test with corrected p-values according to Holm's were used to detect the differences between the 4 treatment modalities.

Results

All animals showed uneventful healing of the area of surgery and no reductions in body weights were noted. Upon specimen retrieval, 3 cylinders were dislocated from the skull bone because of loss of fixation and were embedded in soft connected tissue. These 3 cylinders, 2 test sites and one control site, were excluded from further analysis. The remaining 61 cylinders were found to be stable and in the same position as at placement.

Qualitative histological evaluation revealed varying amounts of newly formed bone with no signs of inflammation in all cylinders. In the empty control cylinders, the augmented tissue comprised of slender bone trabeculae and large marrow spaces. The bone trabeculae adjacent to the surface of the inner wall of the cylinders were oriented parallel to and in various degrees of intimate contact with the surface of the machined cylinders.

The amount of newly formed bone within the control cylinders containing the unfunctionalized PEG matrix and the HA/TCP granules alone varied greatly. In contrast to the empty cylinders, the bone growth was not dominantly along the titanium walls and new bone was mostly in intimate contract with the granulate, which appeared intact and evenly distributed within the augmented tissue. In the upper third of the cylinders, the HA/TCP granules were mainly surrounded by non-mineralized tissue. In the two test groups, significantly more newly formed bone could be detected, partly reaching the upper third of the cylinder.

The area of bone regeneration on the sections of the cylinders was found to be as follows:

Area of bone
regeneration

| Condition | Number of samples | Mean (%) | SE |
|---|---|---|---|
| PEG-PTH 100 | 16 | 53.5 | 5.1 |
| PEG-PTH 20 | 14 | 51.1 | 5.4 |
| PEG | 16 | 34.3 | 5.1 |
| empty | 15 | 23.2 | 5.2 |

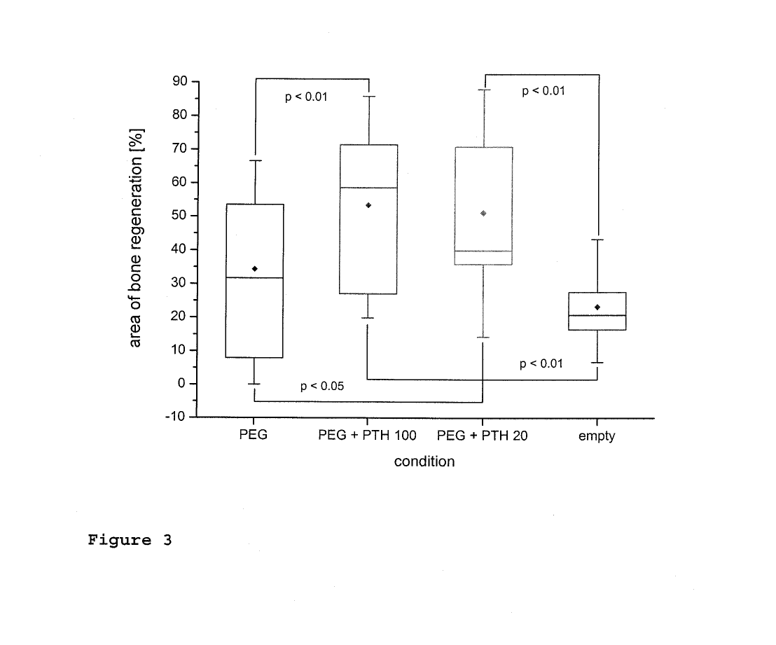

FIG. 3 shows the areas of bone regeneration for the different treatments as well as the significance levels. From these data, it is concluded that the combination of a granulate and a polyethylene glycol hydrogel containing a covalently bound peptide of the parathyroid hormone combined with HA/TCP granules significantly stimulates in situ bone augmentation in rabbits.

Specifically, FIG. 3 shows the area of bone regeneration for the rabbit cranial cylinder model applying granules with PEG. Values are displayed as box-plots ranging from the 25^{th} to the 75^{th} quantiles, including the median and whiskers extending 1.5 times the interquartile range.

### EXAMPLE 5

Methods

8 adult (12 months old) New Zealand White rabbits, weighing between 3 and 4 kg, were anesthetized and obtained each 4 titanium cylinders of 7 mm in height and 7 mm in outer diameter, which were screwed in 1 mm deep circular perforated slits made in the cortical bones of the calvaria. The following 4 treatment modalities were randomly allocated: (1) empty control, (2) a combination of PEG matrix containing 0.31 mg/ml covalently bound RGD and hydroxyapatite (HA)/tricalciumphosphate (TCP) granules (Straumann Bone Ceramic; Institut Straumann AG, Basel, Switzerland), and a combination of PEG matrix containing 0.31 mg/ml covalently bound RGD and either 15 μg (3) or 30 μg (4) of non-bound recombinant BMP-2 and HA/TCP granules.

Immediately before application, 4-arm PEG-acrylate 15 k and HS-PEG-SH 3.4 k (both from Nektar, Huntsville, Ala., USA) were dissolved in 2 mM aqueous HCl solution to yield a homogeneous solution containing equimolar numbers of acrylate and thiol groups, which was then sterile filtered. Aliquots of the sterile PEG solution, a solution of a 9 amino acid cys-RGD peptide (Bachem, Bubendorf, Switzerland), and a BMP-2 solution were combined with a 0.4 M triethanolamine/HCl buffer (pH 8.85) to yield 204 μl of a solution containing 9.8 wt % PEG, 0.31 mg/ml RGD, and 0, 74, or 147 μg/ml BMP-2. This solution was then applied onto 150 mg of HA/TCP granules and mixed for about 10 seconds. Subsequently, this granular putty was applied into the determined cylinders. Within 60 seconds, the PEG gels set and thus stabilized the HA/TCP granules. The cylinders were left open towards the bone side but were closed with a titanium lid towards the covering skin-periosteal flap. The periosteum and the cutaneous flap were adapted and sutured for primary healing.

After 8 weeks, the animals were sacrificed and ground sections were prepared for histology.

The bone formation in the cylinders was evaluated histologically. Mean values and standard deviations were calculated for the amounts of bone formation within the cylinders, either evaluated by the point measurements or by the area of bone regeneration.

Results

All animals showed uneventful healing of the area of surgery and no reductions in body weights were noted.

The area percentages of mineralized bone on the sections of the cylinders were found to be as follows:

Mineralized bone

| Condition | Number of samples | Mean (%) | SD |
|---|---|---|---|
| PEG-BMP 30 μg | 10 | 30.2 | 7.6 |
| PEG-BMP 15 μg | 10 | 25.0 | 7.9 |
| PEG | 10 | 15.2 | 8.0 |
| empty | 9 | 13.9 | 5.7 |

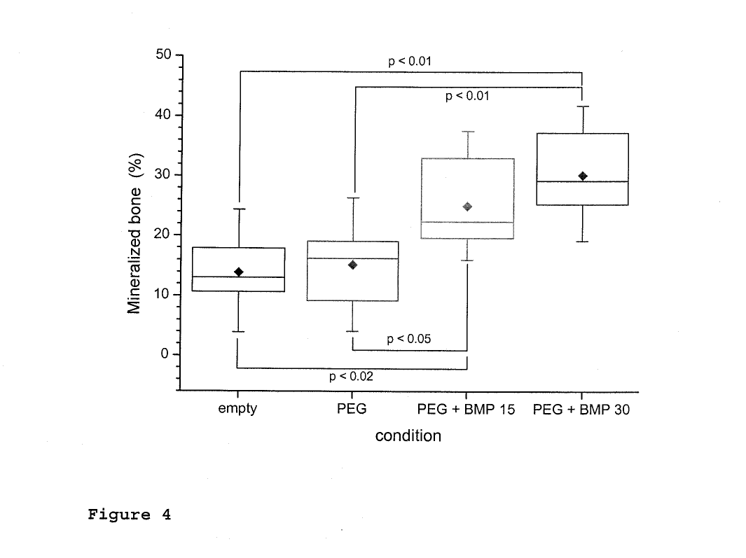

FIG. 4 shows the area percentages of bone regeneration for the different treatments as well as the significance levels. From these data, it is concluded that the combination of a granulate and a polyethylene glycol hydrogel containing a covalently bound RGD peptide and entrapped BMP-2, combined with HA/TCP granules significantly stimulates in situ bone augmentation in rabbits.

Specifically FIG. 4 shows the percentages of mineralized bone found for the rabbit cranial cylinder model applying granules with PEG. Values are displayed as box-plots ranging from the 25^{th} to the 75^{th} quantiles, including the median and whiskers extending 1.5 times the interquartile range.

1. A composition comprising
a granulate selected from the group consisting of autogenous bone material, bone and/or bone like material from natural sources, synthetic materials and mixtures thereof; and
a matrix obtainable by a self selective reaction of at least two precursors A and B in the presence of water, wherein
a first precursor A comprising a core carrying n chains each having a conjugated unsaturated group or a conjugated unsaturated bond attached to any of the last 20 atoms of the chain and
a second precursor B comprising a core carrying m chains each having a thiol or an amine attached to any of the last 20 atoms of the chain, wherein
m is greater than or equal to 2,
n is greater than or equal to 2,
m+n is greater than or equal to 5.

2. Composition according to claim 1, wherein the granulate comprises hydroxyapatite and tricalciumphosphate.

3. Composition according to claim 1, wherein the granulate comprises hydroxyapatite and/or tri-calcium phosphate.

4. Composition according to claim 3, wherein the weight ratio of hydroxyapatite/tricalciumphosphate in the granulate is between 0.1 to 5.0, preferably between 1.0 to 4.0, most preferably between 1.0 to 2.0.

5. Composition according to claim 3, wherein the content of hydroxyapatite in the granulate is at least 1% by weight, preferably equal to or more than 15% by weight, most preferably equal to or more than 50% by weight.

6. Composition according to claim 1, wherein the composition comprises 10% to 80% by weight granulate, preferably 20% to 70%, most preferably 30% to 60%.

7. Composition according to claim 1, wherein the core of precursor A is a carbon atom, a nitrogen atom, ethylene oxide, an amino acid or a peptide, a carbohydrate, a multifunctional alcohol, glycerol or oligoglycerol.

8. Composition according to claim 1, wherein the core of precursor B is a carbon atom, a nitrogen atom, ethylene oxide, an amino acid or a peptide, a carbohydrate, a multifunctional alcohol, glycerol or oligoglycerol.

9. Composition according to claim 7, wherein the core of precursor A is a carbon atom, an ethylene oxide unit, glucose, D-sorbitol, pentaerythritol, glycerol or hexaglycerol.

10. Composition according to claim 8, wherein the core of precursor B is a carbon atom, an ethylene oxide unit, a peptide, glucose, D-sorbitol, pentaerythritol, glycerol or hexaglycerol.

11. Composition according to claim 10, wherein the peptide comprises one or more enzymatic degradation sites.

12. Composition according to claim 1, wherein precursor B comprises a peptide which comprises one or more enzymatic degradation sites.

13. Composition according to claim 1, comprising a third precursor which includes or is a peptide, wherein the peptide includes one or more enzymatic degradation sites.

14. Composition according to claim 1, wherein the conjugated unsaturated group or the conjugated unsaturated bond of first precursor A is an acrylate, an acrylamide, a quinine, a 2- or 4-vinylpyridinium, vinylsulfone, maleimide or an itaconate ester.

15. Composition according to claim 14, wherein the conjugated unsaturated group or the conjugated unsaturated bond of first precursor A is an acrylate.

16. Composition according to claim 1, wherein the first precursor A is selected from the group consisting of

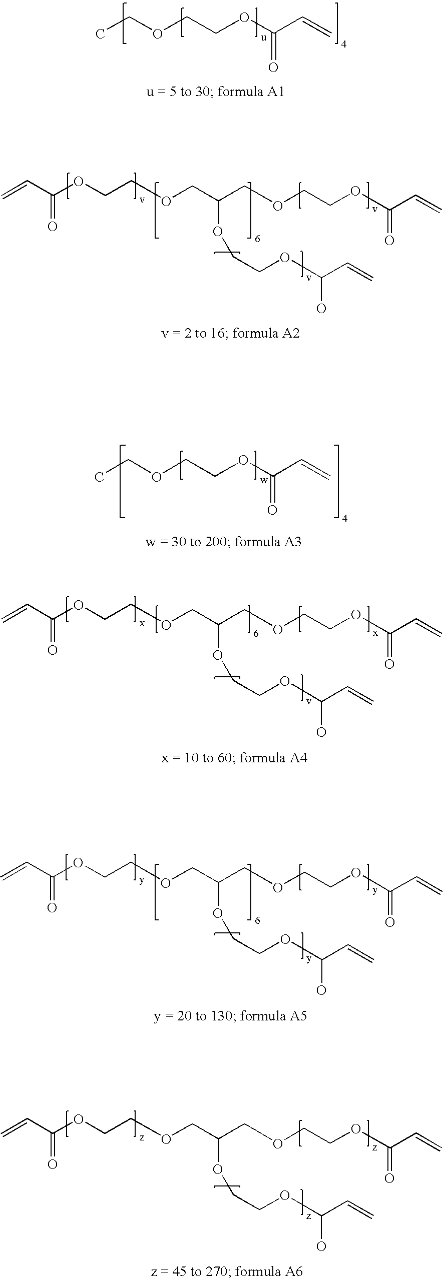

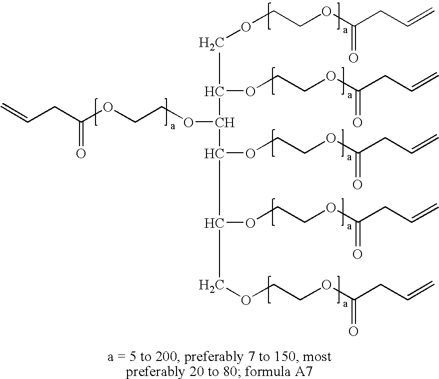

17. Composition according to claim 1, wherein the precursor B comprises a thiol moiety.

18. Composition according to claim 1, wherein the second precursor B is selected from the group consisting of

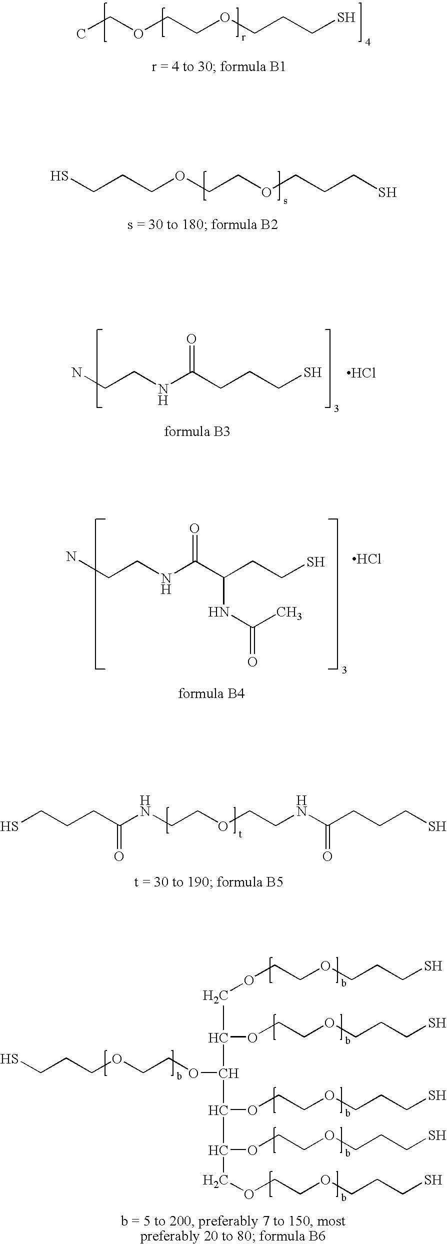

19. Composition according to claim 1, wherein the precursors A and/or B comprise chains having a molecular weight between 500 and 100,000 Da, preferably between 1000 and 50,000 Da, most preferably between 2000 and 30,000 Da.

20. Composition according to claim 1, wherein the chains of precursor A and/or B are a polymer selected from the group consisting of poly(vinyl alcohol), poly(alkylene oxides), poly(ethylene glycol), poly(oxyethylated polyols), poly(oxyethylated sorbitol, poly(oxyethylated glucose), poly(oxazoline), poly(acryloyl-morpholine), poly(vinylpyrrolidone), and mixtures thereof.

21. Composition according to claim 1, wherein the chains of precursor A and/or B are poly(ethylene glycol).

22. Composition according to claim 1, wherein the composition comprises at least one bioactive factor covalently bound to the matrix or entrapped in the composition.

23. Composition according to claim 22, wherein the bioactive factor comprises a thiol.

24. Composition according to claim 22, wherein the bioactive factor is selected from the group consisting of parathyroid hormones (PTH), peptides based on PTH, peptide fragments of PTH, peptides comprising a RGD tripeptide, transforming growth factor beta family (TGFβ), bone morphogenetic protein family (BMP), platelet derived growth factor family (PDGF), vascular endothelial growth factor family (VEGF), insulin like growth factor family (IGF), fibroblast growth factor family (FGF), enamel matrix derivative proteins and peptides (EMD), prostaglandin E₂ (PGE₂) and dentonin.

25. Composition according to claim 24 wherein the bioactive factor is selected from the group consisting of parathyroid hormones (PTH), peptides based on PTH and peptide fragments of PTH.

26. Composition according to claim 24 wherein the bioactive factor is Cys(PTH₁₋₃₄).

27. Composition according to claim 24 wherein the enamel matrix derivative protein or peptide (EMD) is selected from the group consisting of amelogenin, amelin, tuftelin, ameloblastin, enamelin and dentin sialoprotein.

28. Composition according to claim 24, wherein the bioactive factor is H-Gly-Cys-Gly-Arg-Gly-Asp-Ser-Pro-Gly-NH₂.

29. Kit for preparing a composition according to claim 1 comprising
(i) the granulate, and
(ii) the precursor A, and
(iii) the precursor B,
wherein the granulate, precursor A and precursor B are individually stored components.

30. Kit according to claim 29 comprising
(iv) at least one bioactive factor, wherein the bioactive factor is a further individually stored component.

31. Kit according to claim 29, wherein the granulate and precursor A are stored as premix.

32. Kit according to claim 29, wherein the granulate and precursor B are stored as premix.

33. Kit according to claim 30, wherein the precursor B and the bioactive factor are stored as premix.

34. Kit according to claim 30, wherein the bioactive factor is stored in lyophilized form.

35. Kit according to claim 30, wherein the bioactive factor is stored in suitable buffer solution.

36. Method for preparing a composition according to claim 1 by mixing the granulate, the precursor A and the precursor B in the presence of water in a suitable container.

37. Method for preparing a composition according to claim 1 by
a) mixing the precursor A, the precursor B and the bioactive factor in the presence of water in a suitable container, and
b) adding and mixing the granulate with the premix obtained in step a).

38. A method comprising applying a composition according to claim 1 as a bone repair and/or bone augmentation material.
